(19) United States
(12) Patent Application Publication
(10) Pub. No.: US 2026/0233263 A1
(43) Pub. Date: Aug. 13, 2026

(54) SURGICAL ASSEMBLY AND SURGICAL INSTRUMENT

(71) Applicant: Alesi Surgical Limited, Cardiff (GB)

(72) Inventors: Laura Stewart, Cardiff (GB); Ioanna Deroukaki, Cardiff (GB); Dominic Griffiths, Cardiff (GB); Jason Brewer, Cardiff (GB)

(21) Appl. No.: 19/153,477

(22) PCT Filed: Dec. 14, 2023
(86) PCT No.: PCT/GB2023/053247

§ 371 Date: Aug. 4, 2025

(30) Foreign Application Priority Data
Feb. 16, 2023 (GB) 2302221.3

(51) Int. Cl. B08B 6/00; A61B 90/00

(52) U.S. Cl. CPC B08B 6/00; A61B 90/08

(57) ABSTRACT

A surgical assembly and instrument are disclosed for removing particulate matter generated during a surgical procedure. The assembly comprises at least two electrodes, each of the at least two electrodes being electrically couplable with a pole of the same polarity of at least one DC voltage supply. The at least one DC voltage supply is arranged to generate an electrical field from a distal portion of the at least two electrodes for ionizing particulate matter suspended proximate a surgical site. The distal portion of each of the at least two electrodes diverge away from each other in a direction with is toward the longitudinal axis of the tool-piece. The divergent nature of the electrodes is particularly suited to open surgical procedures since the electrodes create a wide electric field for ionizing and capturing particular matter.

The present invention relates to a surgical assembly and a surgical instrument.

Particulate matter in aerosol form is commonly encountered during surgical procedures. For example, it can be either utilized to deliver a therapeutic agent or can be generated as a result of performing a surgical procedure. The generation of particulate matter is typically associated with “energy-based” surgical instruments. Energy-based surgical instruments are powered in some manner in order to deliver a therapeutic effect such as cutting or coagulating patient tissue. Although there are several modes of action such as radiofrequency (RF), ultrasonic and laser, all of these energy-based instruments create particulate matter as a by-product of their mode of action.

The generation of particulate matter obscures the view of the surgeon and is generally harmful if inhaled. Accordingly, there is a desire to remove particulate matter generated during surgical procedures before they can enter the surgical room or otherwise move beyond the surgical site. Historically vacuum-based systems have been used to extract the aerosol particulate matter from the surgical field. However, because this is a dilution-based process it is inefficient at rapidly removing the particulate matter and improving the visual field quality for the surgeon. Such systems require lengths of bulky tubing to be attached to the surgical instrument, adding weight to the assembly which makes it bulky and unwieldy. The tubing is often incorporated into the hand piece of the surgical instrument which can negatively impact the ergonomics and obscure the visibility of the instrument tip and operative site, particularly when performing precise tissue dissection. In addition to this, and in the case of surgical procedures that require gas insufflation to create an operative space, such as laparoscopic surgery for example, the resulting exchange of gas dries and desiccates tissue which has a detrimental effect for the patient. As a result of this and the fact that vacuum-based systems are loud and cumbersome, the adoption of vacuum-based systems has been poor.

WO2011/010148 discloses an alternative approach for managing particulate matter in surgical procedures via an apparatus for the reduction and removal of surgical smoke and other aerosol particulates generated during electrosurgical procedures. The apparatus generates a stream of electrons from a pointed electrode placed near the surgical site, such as within an abdominal cavity, and the electrons emitted from the electrode attach themselves to the aerosol particles suspended nearby. The apparatus further establishes an electrical potential difference between the electrode and the patient for attracting the ionized particles away from the surgical site and thus improving the surgeon's view of the site.

However, the effectiveness of the apparatus is also dependent on the positioning of the electrode relative to the site of surgery and other surgical instruments, and is thus subject to the surgeons experience and skill.

We have now devised a surgical assembly and a surgical instrument which address at least some of the above-mentioned limitations.

According to a first aspect of the invention, there is provided a surgical assembly for removing particulate matter generated during a surgical procedure, the assembly comprising at least two electrodes, each of the at least two electrodes being electrically couplable with a pole of the same polarity of at least one DC voltage supply, the at least one DC voltage supply being arranged to generate an electrical field from a distal portion of the at least two electrodes for ionizing particulate matter suspended proximate a surgical site,
wherein the distal portion of each of the at least two electrodes diverge away from each other in a direction which is toward the longitudinal axis of the tool-piece.

The divergent nature of the electrodes create a wide electric field which is particularly suited for ionizing particulate matter generated in open surgery procedures. The wide electric field increases the ionization zone for particulate matter and reduces any particular matter from escaping from the surgical site. In addition, the use of at least two electrodes enables the surgical instrument to be used in a number of positions and orientations without reducing the particulate clearing effect of the assembly.

The distal portion of each of the at least two electrodes can extend along a plane which is arranged to converge toward a longitudinal axis of a tool-piece of a surgical instrument.

In an embodiment, the at least two electrodes extend in a common plane which is arranged to converge toward the longitudinal axis.

In an embodiment, each plane or the common plane is arranged to converge toward an active region of the tool-piece, which comprises the region that is arranged to deliver energy to patient tissue in performing the surgical procedure. The active region may comprise a distal tip of the tool-piece.

In an embodiment, the common plane is arranged to intersect the active region of the tool-piece.

In an embodiment, the assembly further comprises a cable which is electrically coupled at one end with the at least two electrodes and electrically terminated at an opposing end with a plug for electrically coupling the cable with the at least one DC voltage supply. In an alternative embodiment, the assembly comprises at least two DC voltage supplies, each of the at least two electrodes being electrically couplable with a pole of the same polarity of a respective DC voltage supply.

In an embodiment, the assembly further comprises a body for housing the at least two electrodes. The body may comprise coupling means for detachably coupling the body with a surgical instrument, such as with a handle of the surgical instrument.

In an embodiment, the coupling means permits the body to move relative to the surgical instrument so that the body can be suitably positioned relative to the tool-piece, so that each plane or the common plane along which the distal portion of the at least two electrodes extend converges toward the longitudinal axis of the tool-piece, or intersects the active region.

In an embodiment, the assembly further comprises the tool-piece for the surgical instrument. The tool-piece may be mounted within the body and arranged to form a communicative coupling with an energy source, via the surgical instrument, for performing the surgical procedure.

The energy source may comprise an electrical power source, such as an RF source, for delivering RF power to patient tissue via the tool-piece, an electromagnetic radiation source, such as a laser, for delivering lasing radiation to patient tissue via the tool-piece, a microwave radiation source for delivering microware radiation to patient tissue, or an ultrasonic based energy source for generating ultrasonic vibrations within the tool-piece and delivering ultrasonic vibrations to patient tissue.

The tool-piece may form a communicative coupling with the energy source via a terminal disposed upon the body.

In an embodiment, the at least two electrodes are radially offset from a longitudinal axis of the tool-piece. The at least two electrodes are angularly separated around a longitudinal axis of the tool-piece by less than 180°, and preferably by less than 90° and more preferably by less than 45°. The at least two electrodes may be housed within a body with at least the distal portion of each electrode extending out from the body.

In an embodiment, the distal portion of each of the at least two electrodes are longitudinally spaced from the active region of the tool-piece.

In an embodiment, the electrodes further comprise an insulating sheath extending along at least a portion of the distal portion of the electrodes so that only a distal tip of each electrode is exposed for generating the electric field. Similarly, the tool-piece comprises an insulating sheath extending along at least a portion thereof so that only the active region is exposed. The insulation minimizes the generation of any electrical pathways developing between the tool-piece and the distal portion of the electrodes owing to electrically conductive fluid which is found to precipitate on the electrodes and tool-piece during the surgical procedure. To further mitigate the development of electrical pathways, the insulation may comprise a corrugated or saw-tooth outer profile to maximise the creepage distance between the active region of the tool-piece and the distal portions of the electrodes.

In an embodiment, the assembly further comprises a further electrode which is electrically couplable with a pole of the at least one DC voltage supply which is opposite the pole to which the at least two electrodes are coupled, for attracting ionized particulates. In an embodiment, the further electrode is arranged to form an electrical coupling with the patient undergoing the surgical procedure, so that ionized particulates become attracted toward the patient. The at least two electrodes and the further electrode thus create an electrostatic field to facilitate the electrostatic precipitation of the ionized particulate matter upon the further electrode or upon the patient, for example.

In an embodiment, the assembly further comprises at least one DC voltage supply for delivering DC power to the at least two electrodes and the further electrode. In an embodiment, the DC voltage supply comprises at least two DC voltage supplies, each of the at least two electrodes being electrically couplable with a pole of the same polarity of a respective DC voltage supply. The assembly further comprises an actuator for selectively delivering DC power from the or each DC voltage supply to the at least two electrodes. Alternatively, the assembly may further comprise a sensor for sensing an activation state of the tool-piece, and a controller which is communicatively coupled with the sensor and arranged to receive a sensing signal from the sensor representative of an activation state of the tool-piece, the controller being arranged to synchronize the delivering of DC power to the at least two electrodes in dependence of an activation of the tool-piece.

According to a second aspect of the invention, there is provided a surgical instrument for performing a surgical procedure, the instrument comprising
at least two electrodes, each of the at least two electrodes being electrically couplable with a pole of the same polarity of at least one DC voltage supply, the at least one DC voltage supply being arranged to generate an electrical field from a distal portion of the at least two electrodes for ionizing particulate matter suspended proximate a surgical site,
wherein the distal portion of each of the at least two electrodes diverge away from each other in a direction which is toward the longitudinal axis of the tool-piece.

The distal portion of each of the at least two electrodes can extend along a plane which is arranged to converge toward a longitudinal axis of a tool-piece.

In an embodiment, the instrument further comprises a conduit comprising a cable which is electrically coupled at one end with the at least two electrodes, the conduit further comprising a channel for communicating energy from an energy source to the tool-piece of the instrument, the conduit being terminated at a plug for electrically coupling the cable with the at least one DC voltage supply and for coupling the tool-piece with the energy source.

In an embodiment, the surgical instrument comprises a handle and the tool-piece is detachably couplable with the handle so that different tool-pieces may be used for different surgical procedures.

In an embodiment, the tool-piece comprises an active region for delivering energy to patient tissue in performing the surgical procedure.

Embodiments of the present invention will now be described by way of example only and with reference to the accompanying drawings in which:

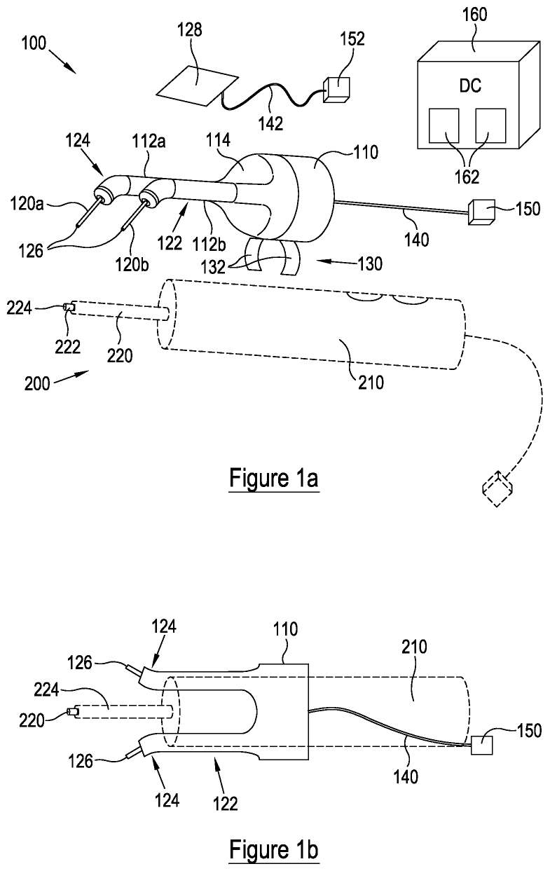

FIG. 1a is a schematic illustration of a surgical assembly according to a first embodiment of the present invention;

FIG. 1b is a plan view of the surgical assembly illustrated in FIG. 1a;

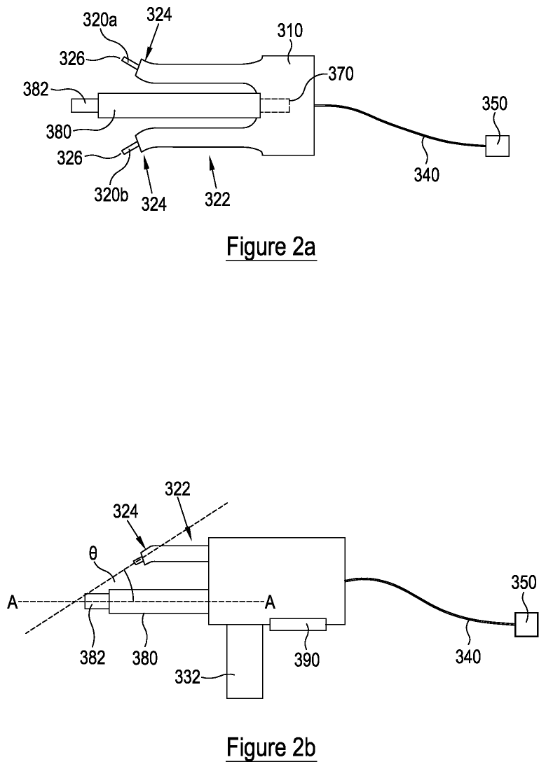

FIG. 2a is a plan view of a surgical assembly according to a second embodiment of the present invention;

FIG. 2b is a side view of the surgical assembly illustrated in FIG. 2a;

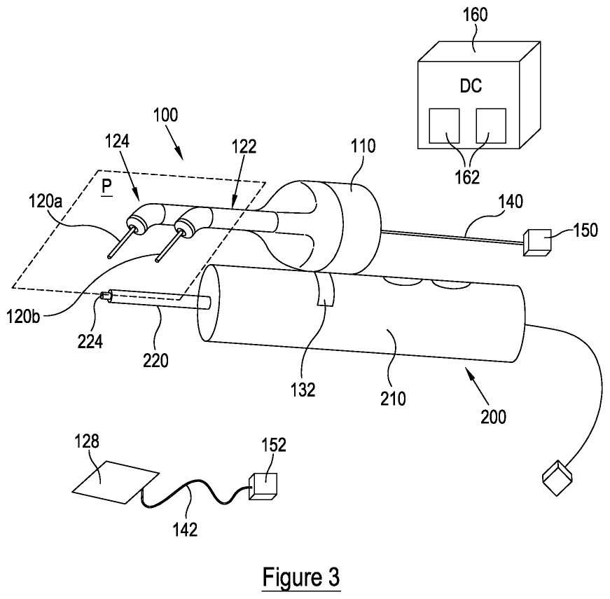

FIG. 3 is a schematic illustration of a surgical assembly illustrated in FIG. 1a mounted upon a surgical instrument;

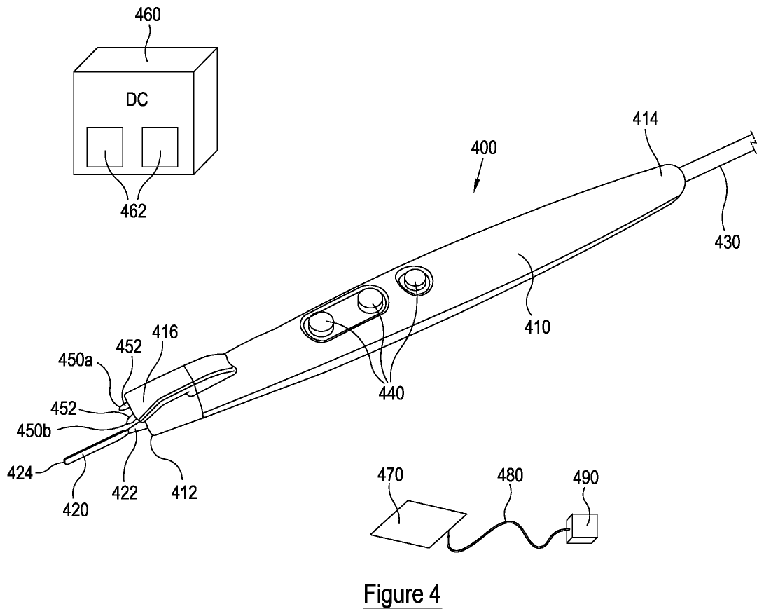

FIG. 4 is a perspective view of a surgical instrument according to an embodiment of the present invention;

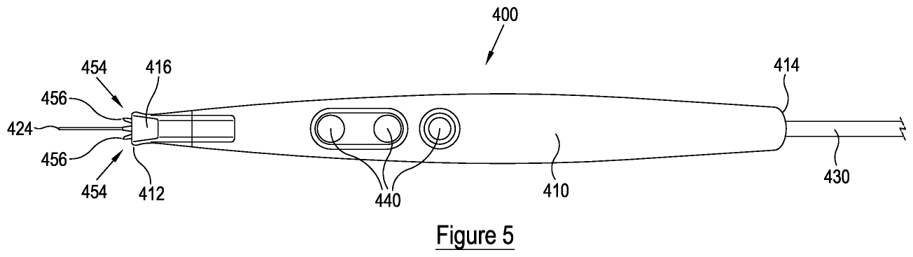

FIG. 5 is a plan view of the surgical instrument illustrated in FIG. 3; and,

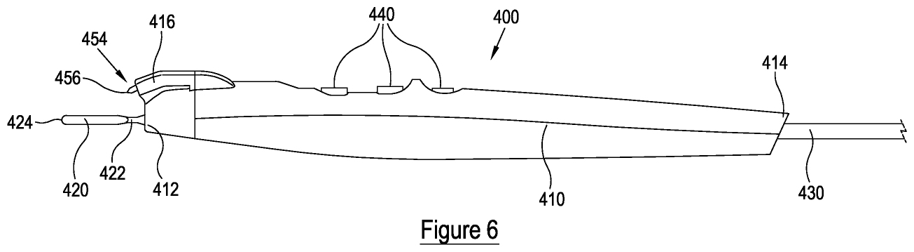

FIG. 6 is a side view of the surgical instrument illustrated in FIG. 3.

Referring to FIG. 1a of the drawings there is illustrated a surgical assembly 100 according to a first embodiment of the present invention for use in clearing particulate matter, particularly aerosolized matter, generated at a surgical site during a surgical procedure. The assembly 100 is arranged to retrofit to a surgical instrument 200 and comprises a generally cylindrical body 110 formed of a rigid plastics material (although other body shapes could be used) for supporting two electrodes 120a, 120b, although three or more electrodes may equally be employed. The body 110 comprises two sleeves 112a, 112b formed of an electrically insulating material which extend forwardly from a front end face 114 of the body 110. The sleeves 112 and body 110 may be formed integrally and each sleeve 112 a, 112 b is arranged to receive and support a respective electrode 120a, 120b. The body 110 further comprises coupling means 130, which may comprise resiliently deformable clips 132 which are arranged to “snap-locate” around a handle portion 210 of a surgical instrument 200 for example, for mounting the body 110 to the instrument 200.

A proximal portion 122 of each electrode 120a, 120b, namely the portion arranged closest to the body 110, extend in a substantially parallel arrangement within the respective sleeve 112a, 112b, whereas a distal portion 124 of each electrode 120a, 120b diverge away from each other in a direction which is away from the front end face 114 of the body 110. The distal portion 124 of each electrode 120a, 120b extend along a common plane and when the body 110 is fitted to a surgical instrument 200 the common plane is arranged to converge toward an active region 222 of a tool-piece 220 associated with the surgical device 200. In this respect it is evident that the distal portion 124 of each electrode 120a, 120b extends in a different plane compared with the proximal portion 122. The insulating sleeves 112a, 112b extend along the proximal portion 122 of each electrode 120a, 120b and along a portion of the distal portion 124 of each electrode 120a, 120b, and may comprise a corrugated or saw-tooth outer profile (not shown) to increase the creepage distance between the distal portion 124 of the electrodes 120 and the active region 222. However, the distal region or tip 126 of each electrode is exposed.

The active region 222 of the tool-piece 220 is the region of the tool-piece 220 which is arranged to contact patient tissue and deliver the desired therapeutic effect, such as cutting, cauterizing or coagulating patient tissue, and typically comprises a distal tip 224 of the tool-piece 220.

The assembly 100 further comprises a cable 140 which is electrically coupled at one end with each electrode 120 while the opposing end of the cable 140 is terminated at a plug 150 which is receivable within a socket 162 of a DC voltage supply 160 for electrically coupling each electrode 120 with a common pole of the DC voltage supply 160, such as the negative pole. In an alternative embodiment, the assembly comprises at least two DC voltage supplies (not shown) and each electrode is electrically couplable with a pole of the same polarity, such as the negative pole of a respective DC voltage supply, such that each electrode 120 is driven by a respective DC supply 160. In this respect, it is envisaged that the assembly may comprise a cable 140 having at least two plugs 150 for electrically coupling each electrode 120a, 120b with a respective DC voltage supply 160.

The assembly 100 further comprises an arrangement of resistors (not shown) which may be disposed within the electrical pathway between the electrodes 120 and the cable 140 for limiting capacitance associated with the assembly 100 and the energy associated with an electrostatic discharge from the electrodes 120 back to the voltage supply 160. The resistor arrangement may be disposed within the body 110 or within the or each DC voltage supply 160.

The assembly 100 further comprises a further electrode for attracting ionized particulate matter away from the surgical site. The further electrode is electrically couplable with an opposing pole of the or each DC voltage supply 160, such as a positive pole (or earth) via the cable 140, and may comprise a metal collector plate or gauze (not shown) which is placed in proximity with the surgical site, or a metal collar (not shown) which may be disposed on the surgical instrument 200 for example. Alternatively, the further electrode may comprise an adhesive pad 128 for forming an electrical coupling with the patient undergoing the surgical procedure. The pad 128 is electrically couplable with an opposing pole of the (or each) DC voltage supply 160, such as a positive pole via a further cable 142 and plug 152 for coupling with a socket 162 on the DC supply. The at least two electrodes 120a, 120b, the further electrode 128 and the (or each) DC voltage supply 160 thus serve to create an electrostatic field between the patient (not shown) and the electrodes 120a, 120b to facilitate the ionization and electrostatic precipitation of the particulate matter upon the patient. The electrical coupling of the or each DC voltage supply 160 to the electrodes 120a, 120b and further electrode 128 is controlled by an actuator (not shown), which may comprises a switch disposed on the DC voltage supply 160 or a footswitch that is electrically coupled with the or each DC voltage supply 160. In this respect an operator, such as a surgeon can choose to selectively supply DC electrical power to the electrodes 120a, 120b and further electrode 128 when it is required to clear the view of the surgical site. Alternatively, the assembly 100 may further comprise a sensor (not shown) for sensing an activation state of the tool-piece 220, and a controller (not shown) which is communicatively coupled with the sensor and arranged to receive a sensing signal from the sensor representative of an activation state of the tool-piece, the controller being arranged to synchronize the delivering of DC power 160 to the electrodes 120a, 120b in dependence of an activation of the tool-piece 220.

Referring to FIG. 2, there is illustrated a surgical assembly 300 according to a second embodiment of the present invention. The assembly 300 of the second embodiment is substantially similar to the assembly of the first embodiment and so like features have been referenced with the same numerals but increased by 200.

The assembly 300 of the second embodiment however, further comprises a port 370 located within the body 310 for receiving a tool-piece 380 which is used for performing the surgical procedure. The tool-piece 380 may comprise a metal blade for delivering RF electrical power from an RF source (not shown) to patient tissue, or a waveguide for delivering lasing radiation from a source of lasing radiation (not shown). Alternatively, the tool-piece 380 may comprise a blade arranged in vibrational communication with a piezoelectric element (not shown) which is arranged to deliver ultrasonic energy to patient tissue. The tool-piece 380 is arranged to detachably couple with the body 310 so that different tool-pieces 380 may be used for different procedures. The tool-piece 380 is receivable or arranged to dock within the port 370 disposed on a front face 314 of the body 310 such that the tool-piece 380 extends forwardly of the body 310 substantially parallel with the proximal portion 322 of the electrodes 320a, 320b. The portion of the tool-piece 380 which is received within the port 370 forms a communicative coupling with a terminal 390 formed on the body 310. The terminal 390 is arranged to form a coupling with an adjacent terminal (not shown) on the handle 210 of a surgical instrument 200 when the body 310 is secured thereto, so that energy from an energy source (not shown), such as an electrical RF source, a lasing radiation source or an ultrasonic energy source, may be communicated to the tool-piece 380 from the instrument 200.

Referring to FIGS. 1-3 of the drawings, in both the first and second embodiments of the assembly, the proximal portion 122, 322 of each electrode 120, 320 is radially spaced from a longitudinal axis A of the tool-piece 220, 380 and the distal portion 124, 324 of each electrode 120, 320 is longitudinally spaced from the active region 222, 382 of the tool-piece 220, 380 such that the common plane P (as illustrated in FIG. 3 of the drawings) forms an acute angle θ with the longitudinal axis A. The radial and longitudinal spacing serves to maintain a desired spacing between the distal portion 124, 324 of each electrode 120, 320 and the active region 222, 382 to ensure a sufficient potential difference between the distal portion 124, 324 of the electrodes 120, 320 and the patient to achieve a suitable ionization of particulate matter. Moreover, the spacing serves to minimize any electrical pathways developing between the distal tip 126 of each electrode 120a, 120b and the active region 222, 382 of the tool-piece 220, 380, owing to the deposition of (electrically conducting) fluid thereon during the surgical procedure, which would other create a direct electrical short between the electrodes 120, 320 and the tool-piece 220, 380, for example.

Referring to FIGS. 4-6 of the drawings, there is illustrated a surgical instrument 400 according to an embodiment of the present invention. The instrument comprises a linear, elongate handle 410 comprising a front end 412 and a rear end 414, via which a surgeon can grip and manipulate the instrument 400 in performing a surgical procedure. The instrument 400 further comprises a tool-piece 420 which extends forwardly from the front end 412 of the instrument and which is orientated to extend along an axis which is substantially parallel with an axis of the handle 410. The tool-piece 420 may be detachably couplable with the handle 410 and is arranged to deliver energy to patient tissue in delivering the desired therapeutic effect, from an energy source (not shown). The tool-piece 420 may comprise a metal blade for delivering RF electrical power from an RF source (not shown) to patient tissue, or a waveguide for delivering lasing radiation from a source of lasing radiation (not shown). Alternatively, the tool-piece 420 may comprise a blade arranged in vibrational communication with a piezoelectric element (not shown) for driving the blade to deliver ultrasonic energy to patient tissue. Accordingly, the energy source may comprise an electrical RF source, a lasing radiation source, a source of microwave radiation or an ultrasonic energy source for example, and this is provided to the instrument via a conduit 430 which is coupled to the rear 414 of the handle 410. The conduit 430 may comprise an electrical cable for delivering RF electrical power or a waveguide, such as an optical fibre, for delivering lasing or microwave radiation. Alternatively, the conduit may comprise a cable for delivering electrical power for a piezoelectric element which is used to drive the tool-piece into various vibrational states for delivering ultrasonic vibrations. An opposing end of the conduit is terminated at a plug (not shown) for coupling to the respective energy source, and the energy supply to the tool-piece is controlled using buttons 440 located upon an upper region of the handle 410.

The surgical instrument 400 further comprises two electrodes 450a, 450b which extend from a body 416 of the handle 410 which is disposed proximate the front end 412 of the handle 410. The electrodes 450 comprise an electrically insulating sleeve 452 which extend along at least a portion the length of the electrodes 450, and preferably along at least a portion of the distal portion 454 of each electrode, so that only a distal region or tip 456 of each electrode 450 is exposed. The tool-piece 420 similarly comprises an electrically insulating sleeve 422 which extends along at least a portion thereof such that only an active region 424, namely the region which is arranged to deliver the energy to the patient tissue, is exposed.

The electrodes 450 extend forwardly of the body 410 and the distal portion 454 of each electrode 450 diverge away from each other forwardly of the body 410, toward the active region 424 of the tool-piece 420. Moreover, the distal portion 454 of each electrode 450 further extend along a common plane which converges toward the active region 424 of a tool-piece 420. The distal portion 454 of each electrode 450 is radially spaced from a longitudinal axis of the tool-piece 420 and also longitudinally spaced from the active region 424. In this respect, the distal portion 454 of each electrode 450 extend in a plane which is angled toward the active region 424 of the tool-piece 420 and which forms an acute angle with a longitudinal axis of the tool-piece 420.

The electrodes 450 are arranged to form an electrical coupling with a pole having the same polarity of at least one DC voltage supply 460, such as the negative pole, via a cable which extends within the conduit 430. In this respect, the electrodes 450 may be coupled with a common pole of a single DC voltage supply 460 or electrically coupled with a pole of the same polarity of a respective DC voltage supply (only one of which is illustrated) 460, such that each electrode 450 is driven by a separate DC voltage supply 460. The cable terminates in the plug (not shown) associated with the conduit 430 and as such, the common plug is arranged to deliver power to both the electrodes 450a, 450b and the tool-piece 420 from the respective source. The delivery of DC power to the electrodes 450 may be controlled using buttons 440 located on the handle 410 or may be automated in dependence upon an activation status of the tool-piece 420. For example, the instrument 400 may further comprise a sensor (not shown) for sensing an activation state of the tool-piece 420, and a controller (not shown) which is communicatively coupled with the sensor and arranged to receive a sensing signal from the sensor representative of an activation state of the tool-piece 420, the controller being arranged to synchronize the delivering of DC power to the electrodes 450 in dependence of an activation of the tool-piece 420.

The surgical instrument 400 further comprises a further electrode for attracting ionized particulate matter away from the surgical site. The further electrode is electrically couplable with an opposing pole of the or each DC voltage supply 460, such as a positive pole (or earth) via the cable, and may comprise a metal collector plate or gauze (not shown) which is placed in proximity with the surgical site, or a metal collar (not shown) which may be disposed on the surgical instrument 400 for example. Alternatively, the further electrode may comprise an adhesive pad 470 for forming an electrical coupling with the patient undergoing the surgical procedure. The pad 470 is electrically couplable with an opposing pole of the at least one DC voltage supply 460, such as a positive pole via a further cable 480 and a plug 490 disposed at the end of the cable 480 for coupling with a socket 462 on the DC supply 460. The at least two electrodes 450a, 450b and the further electrode 470 thus create an electrostatic field to facilitate the ionization and electrostatic precipitation of the particulate matter upon the further electrode 470 or patient for example.

The instrument 400 further comprises an arrangement of resistors (not shown) which may be disposed within the electrical pathway between the electrodes 450 and the conduit 430 for limiting capacitance within the instrument 400 and thus reducing the energy associated with an electrostatic discharge from the electrodes 450 back to the or each DC voltage supply 460. The arrangement of resistors may be located within the body 416 of the handle 410, the handle 410 itself, or even within the or each DC voltage supply 460.

When using the surgical assembly 100 illustrated in FIG. 1, the body 110 is coupled to the handle 210 of the instrument 200 and may be repositioned relative to the handle to ensure that the plane within which the distal portion 124 of each electrode 120 extends, intersects the active region 222 of the tool-piece 220. Alternatively, the distal portion 124 of each electrode 120 may be repositioned to extend within the plane that intersects the active region 222. However, when using the surgical assembly 100, 300 of the first or second embodiment, or the surgical instrument 400, the divergent nature of the distal portion of the electrodes 120, 320, 450 facilitates the release of electrons therefrom along a wide arcuate range. Moreover, this arcuate range is arranged directed toward the active region 222, 382, 424 and thus the surgical site to effectively flood the surgical site with low energy gas ions. The gas ions attach themselves to particulate matter suspended near the surgical site and thus become electrostatically charged. However, the potential difference between the electrodes 120, 320, 450 and the patient, or between the electrodes 120, 320, 450 and the further electrode 128, 328, 470, owing to the electrical coupling of the electrodes and patient/further electrode to opposing poles of the or each DC voltage supply 160, 360, 460, causes the electrostatically charged particulates to become readily attracted to the patient tissue and thus vacate the surgical site to maintain a clear visual field for the surgeon. The divergent nature of the distal portion 124, 324, 454 of the electrodes 120, 320, 450 and the convergent nature of the plane within which the distal portion 124, 324, 454 of the electrodes 120, 320, 450 extend toward the active region 222, 382, 424 provides for an effective capture of particulates, such as surgical smoke, before they can be released into the surgical environment. This arrangement of electrodes 120, 320, 450 is thus particularly suited to open surgery procedures which do not have the benefit of an enclosed cavity, such as with laparoscopic procedures to maintain particulates.

1. A surgical assembly for removing particulate matter generated during a surgical procedure, the assembly comprising at least two electrodes, each of the at least two electrodes being electrically couplable with a pole of the same polarity of at least one DC voltage supply, the at least one DC voltage supply being arranged to generate an electrical field from a distal portion of the at least two electrodes for ionizing particulate matter suspended proximate a surgical site,
wherein the distal portion of each of the at least two electrodes diverge away from each other and are arranged to extend along a plane which is arranged to converge toward a longitudinal axis of a tool-piece of a surgical instrument, and wherein each of the at least two electrodes comprises a respective insulating sheath extending along at least a portion of the distal portion of the respective electrode so that only a distal tip of each electrode is exposed for generating the electric field.

2. The surgical assembly according to claim 1, wherein the distal portion of each of the at least two electrodes extend in a common plane which is arranged to converge toward the longitudinal axis.

3. The surgical assembly according to claim 2, wherein each plane or the common plane is arranged to converge toward an active region of the tool-piece which comprises the region of the tool-piece which is arranged to deliver energy to patient tissue in performing the surgical procedure.

4. The surgical assembly according to claim 3, wherein the active region comprises a distal tip of the tool-piece.

5. The surgical assembly according to claim 1, further comprising a cable which is electrically coupled at one end with the at least two electrodes and electrically terminated at an opposing end with a plug for electrically coupling the cable with the at least one DC voltage supply.

6. The surgical assembly according to claim 1, further comprising a body for housing the at least two electrodes.

7. The surgical assembly according to claim 6, wherein the body comprises coupling means for detachably coupling the body with the surgical instrument.

8. A surgical assembly according to claim 7, wherein the coupling means permits the body to move relative to the surgical instrument so that the body can be suitably positioned relative to the tool-piece, so that each plane along which the distal portion of the at least two electrodes extend converges toward the active region of the tool-piece.

9. (canceled)

10. (canceled)

11. (canceled)

12. (canceled)

13. The surgical assembly according to claim 1, wherein the at least two electrodes are radially offset from a longitudinal axis of the tool-piece.

14. The surgical assembly according to claim 1, wherein the at least two electrodes are angularly separated around a longitudinal axis of the tool-piece by less than 180°.

15. The surgical assembly according to claim 1, wherein the distal portion of each of the at least two electrodes is longitudinally spaced from the active region of the tool-piece.

16. (canceled)

17. The surgical assembly according to claim 1, further comprising a further electrode which is electrically couplable with a pole of the DC voltage supply which is opposite the pole to which the at least two electrodes are coupled, the further electrode being arranged to attract ionized particulates from the surgical site.

18. The surgical assembly according to claim 1, further comprising at least one DC voltage supply for delivering DC power to the at least two electrodes.

19. The surgical assembly according to claim 1, further comprising at least two DC voltage supplies, each of the at least two electrodes being electrically couplable with a pole of the same polarity of a respective DC voltage supply.

20. The surgical assembly according to claim 1, further comprising an actuator for selectively delivering DC power from the at least one/two DC voltage supply/supplies to the at least two electrodes.

21. The surgical assembly according to claim 1, further comprising a sensor for sensing an activation state of the tool-piece, and a controller which is communicatively coupled with the sensor and arranged to receive a sensing signal from the sensor representative of an activation state of the tool-piece, the controller being arranged to synchronize the delivering of DC power to the at least two electrodes in dependence of an activation of the tool-piece.

22. A surgical instrument for performing a surgical procedure, the instrument comprising a tool-piece and at least two electrodes, each of the at least two electrodes being electrically couplable with a pole of the same polarity of at least one DC voltage supply, the at least one DC voltage supply being arranged to generate an electrical field from a distal portion of the at least two electrodes for ionizing particulate matter suspended proximate a surgical site,
wherein the distal portion of each of the at least two electrodes diverge away from each other and are arranged to extend along a plane which is arranged to converge toward the longitudinal axis of the tool-piece,
and wherein each of the at least two electrodes comprises a respective insulating sheath extending along at least a portion of the distal portion of the respective electrode so that only a distal tip of each electrode is exposed for generating the electric field.

23. The surgical instrument according to claim 22, wherein the instrument further comprises a conduit comprising a cable which is electrically coupled at one end with the at least two electrodes, the conduit further comprising a channel for communicating energy from an energy source to the tool-piece of the instrument.

24. The surgical instrument according to claim 23, wherein the conduit is terminated at a plug for electrically coupling the cable with the at least one DC voltage supply and for coupling the tool-piece with the energy source.

25. (canceled)

26. (canceled)

27. The surgical instrument according to claim 22, wherein each of the at least two electrodes is electrically couplable with a pole of the same polarity of a respective DC voltage supply.

28. (canceled)

29. (canceled)
